(19) United States
(12) Plant Patent Application Publication
(10) Pub. No.: US 2013/0125277 P1
(43) Pub. Date: May 16, 2013

(54) Phalaenopsis orchid plant named 'Hoodooa'

(50) ;

(71) Applicant: Floricultura, Heemskerk (NL)

(72) Inventor: René Schoone, Assendelft (NL)

(73) Assignee: Floricultura, Heemskerk (NL)

(21) Appl. No.: 13/573,499

(22) Filed: Sep. 19, 2012

Related U.S. Application Data
(60) Provisional application No. 61/540,407, filed on Sep. 28, 2011.

(30) Foreign Application Priority Data
Sep. 30, 2011 (NL) PBR OPS841

(51) Int. Cl. A01H 5/00

(52) U.S. Cl. PLT/311

(57) ABSTRACT

A new and distinct Phalaenopsis plant named ‘Hoodooa’ particularly characterized by flowers which are are white; the labellum is white and yellow with orange/red stripes/spots; plants which may be propagated economically and uniformly using tissue culture; plants which produce more than one inflorescence; long and sturdy inflorescences; and relatively short, dark-green foliage.

### CROSS-REFERENCE TO RELATED APPLICATIONS

This application claims priority to U.S. Provisional Application No. 61/540,407 filed Sep. 28, 2011, and Netherlands Plant Breeders' Rights application no. OPS841 filed Sep. 30, 2011. The disclosures of both prior applications are hereby incorporated by reference.

### LATIN NAME OF THE GENUS AND SPECIES OF THE PLANT CLAIMED

Phalaenopsis hybrida

### VARIETY DENOMINATION

‘Hoodooa’

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Hoodooa’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Hoodooa’ is a product of a controlled breeding program conducted by the inventor, René Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Hoodooa’ originated from a cross made by the inventor in 1999 in Strengweg, Heemskerk, The Netherlands. The female or seed parent is the Phalaenopsis cultivar designated ‘Winter Kaala×amabilis’, unpatented. The male or pollen parent is the Phalaenopsis cultivar designated ‘World Class’, unpatented. The new Phalaenopsis ‘Hoodooa’ was discovered and selected by the inventor as a single flowering plant within the progeny of the stated cross in a controlled environment in 2006 in Strengweg, Heemskerk, The Netherlands.

Asexual reproduction of the new Phalaenopsis cultivar by tissue culture was first performed in July, 2007 in Cieweg 13, Heemskerk, The Netherlands, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘Hoodooa’, which in combination distinguish this Phalaenopsis as a new and distinct cultivar:
1. flowers which are white; the labellum is white and yellow with orange/red stripes/spots;
2. plant produces more than one inflorescence;
3. plants may be propagated economically and uniformly using tissue culture;
4. inflorescence is long and sturdy
5. relatively short, dark-green foliage
   Presently, there is no commercial cultivar to which ‘Hoodooa’ can be meaningfully compared.

### BRIEF DESCRIPTION OF THE PHOTOGRAPHS

The accompanying photographs illustrate the overall appearance of the new Phalaenopsis ‘Hoodooa’ showing the colors as true as is reasonably possible with colored reproductions of this type. Colors in the photographs may differ slightly from the color values cited in the detailed botanical description, which accurately describe the color of ‘Hoodooa’.

FIG. 1 shows a side view perspective of a typical flowering plant of ‘Hoodooa’ in a 12 cm pot, at 16 months of age.

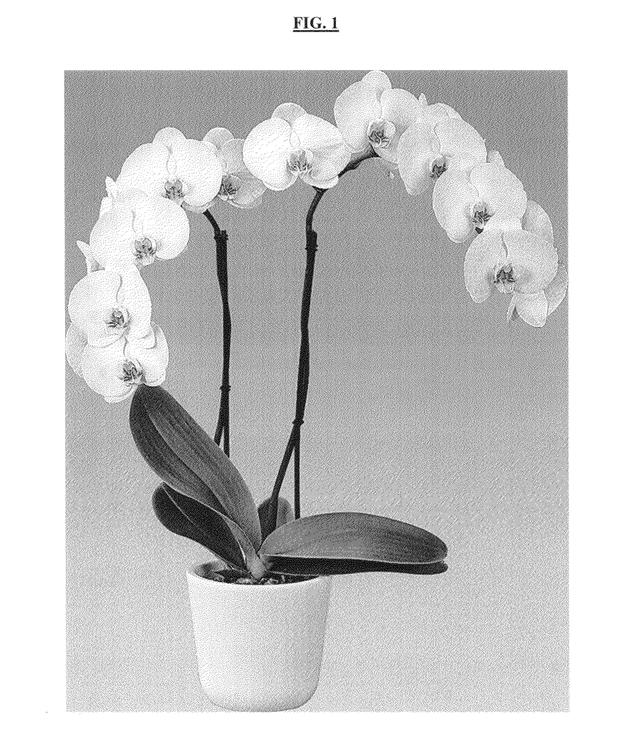

FIG. 2 shows a close-up view of the typical buds and flowers of ‘Hoodooa’.

FIG. 3 shows a close-up view of the typical leaves of ‘Hoodooa’.

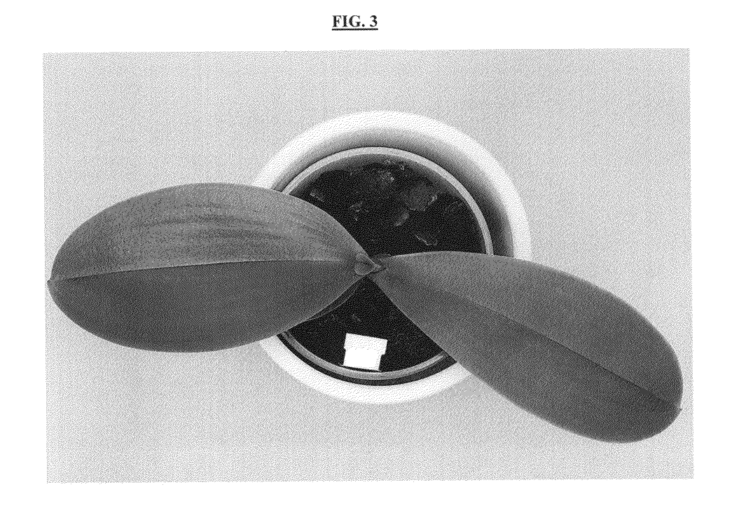

### DETAILED BOTANICAL DESCRIPTION

The new Phalaenopsis cultivar ‘Hoodooa’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘Hoodooa’ as grown in a greenhouse in Strengweg, Heemskerk, The Netherlands, under conditions which closely approximate those generally used in commercial practice. Initially, the ideal temperature to grow plants of ‘Hoodooa’ is 27° C. during the day and at night. Then, during the flowering phase of ‘Hoodooa’, the ideal growing temperature is 20-22° C. during the day and 18° C. at night. Light levels for growing ‘Hoodooa’ are a minimum of 5,000 lux and a maximum of 10,000 lux. A balanced fertilizer with level of 200 ppm N, 87 ppm P, 168 ppm K is applied. Duration of growth of ‘Hoodooa’ from potting size is between 10 and 14 months.

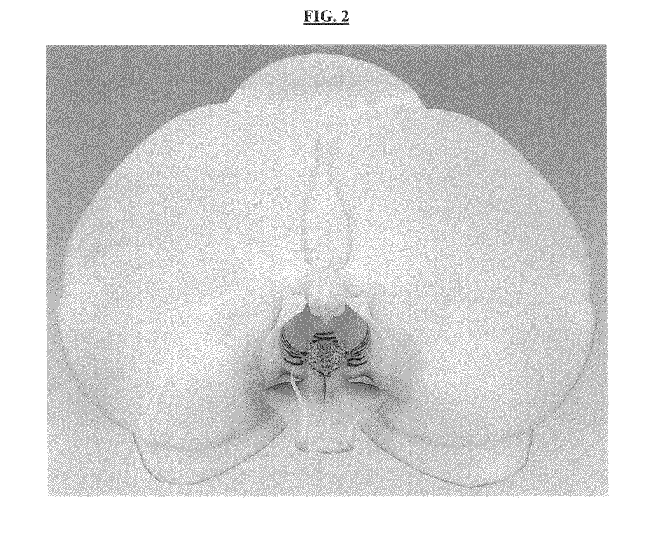

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Hoodooa’ plants described is 12 months after potting.
Classification:
   Botanical.—Phalaenopsis.
Parentage:
   Female or seed parent.—Phalaenopsis cultivar designated ‘Winter Kaala×amabilis’, unpatented.
   Male or pollen parent.—Phalaenopsis cultivar designated ‘World Class’, unpatented.
Propagation:
   Type.—tissue culture.
   Rooting habit and description.—Fleshy; approximately 4 mm-7 mm wide and green/yellow in color (RHS 147B); freely branching. It takes 12 weeks for plants growing in tissue culture to initiate roots.
Plant:
   Size at maturity.—Height: about 62 cm Spread: about 60 cm.
   Growth habit.—standard; dark-green leaves and relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, tip is blunt and asymmetric.
   Texture.—smooth and leathery.
   Pubescence.—3 to 4 pairs of leaves.
   Mature leaf length.—about 14 to 20 cm.
   Mature leaf width.—about 7 to 10 cm.
   Mature leaf thickness.—about 1.5 mm.
   Mature leaf color (upper surface).—RHS N137A.
   Mature leaf color (under surface).—RHS 146A.
   Venation.—Pattern: parallel Color of midvein: upper surface RHS N137A, under surface RHS 146A.
Peduncle:
   Quantity per plant.—about 1 to 2.
   Number of flowers per peduncle.—about 7 to 10.
   Length.—about 70 cm.
   Diameter.—about 6 mm.
   Strength.—strong.
   Aspect.—upright.
   Texture.—glabrous and smooth.
   Color.—RHS 146B.
   Internode.—Length: about 30 to 40 mm.
Inflorescence description:
Appearance: Upright to slightly pendant, racemose inflorescence with bilaterally symmetrical flowers that open in succession beginning with the lowermost flower.
Inflorescence size:
   Height (from base to tip).—about 15 to 25 mm.
   Diameter (at midpoint).—about 12 to 22 mm.
   Shape.—egg-shaped.
Flowering time: For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
Flowering longevity: On the plant: about 4 to 6 months; lastingness of cut flowers: has not been observed
Fragrance: No fragrance
Flower:
   Rate of opening.—Flowers fully opened about 2 to 3 days after tepal separation.
   Orientation at opening.—slanted upward and outward.
   Shape.—see FIG. 2.
   Size.—Height: about 88 mm Diameter: about 99 mm Depth of tube: about 10 mm.
Tepals:
   Quantity and arrangement.—six tepals that are trimerous, overlapping and arranged in 2 whorls. Petals are more pronounced than sepals.
Petals:
   Arrangement.—Inner whorl of tepals comprises 3 petals, 2 lateral petals and labellum.
   2 lateral petals.—Overall shape: broadly ovate and weakly cupped Apex: oval Margin: weakly undulate Length: about 65 mm Width: about 45 mm Texture: Upper surface: smooth and satiny Under surface: smooth and satiny Color (when fully opened): upper surface and under surface are RHS NN155C, white Labellum: Overall shape: 3-lobed with 2 prominent callosities at central junction of the lateral lobes and base of the midlobe. Lateral lobes of labellum fold upward about the column; the midlobe extends forward and is terminated by 2 short filiform appendages at the apex. Lateral lobes of the labellum are ovate in shape while the midlobe is triangular with a bump and a rib on it Margin: entire and weakly undulate Length: about 21 mm Width (not flattened): about 23 mm Texture: Upper surface: smooth and satiny Color (when fully opened): Main color midlob is white (RHS NN155C), at the base orange/red (RHS 34B) extension to yellow (RHS 7A). Chirri is white (RHS NN155C). Under surface at the base white (RHS NN155C), on the sides yellow (RHS 6B) with red/orange edges (RHS 34B). Main color lateral lobs is white (RHS NN155C), at the bottom orange/red (RHS 34B) extension to yellow (RHS 7A). Stripes orange/red (RHS N34A and 34B). Under surface is white (RHS NN155C) with bottom edges orange/red (RHS 34B and yellow (RHS 7A) Chirri: long Pestle (callosities): Length: about 6 mm Width (not flattened): about 8 mm Height: about 7 mm Color: Main color is yellow RHS 9C with RHS 17A. Stripes and spots are red/orange RHS N34A.
Sepals:
   Arrangement.—Outer whorl of tepals comprises 3 sepals.
   Overall shape.—elliptical and weakly cupped Length: about 45 to 55 mm Width: about 33 to 43 mm Texture: Upper & under surface: smooth and satiny Color (when fully opened): Upper surface: white (RHS NN155C), Under surface: white (RHS NN155C with green/yellow haze (RHS 1D).
Reproductive organs:
Arrangement: The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
Column:
   Length.—about 9 mm.
   Diameter.—about 7 mm.
   Color.—white RHS NN155C.
Pollinia:
   Quantity.—Two.
   Size.—about 1 mm.
   Color.—yellow/orange (RHS 23A).
Ovary:
   Length.—about 4 mm.
   Diameter.—about 5 mm.
Pedicel:
   Length.—about 30 to 40 mm.
   Diameter.—about 4 mm.
   Texture.—glabrous and smooth.
   Color.—At the base yellow/green (RHS 146D) and then RHS 145D.
Disease/pest resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Hoodooa’, as illustrated and described herein.
